(19) United States
(12) Patent Application Publication
(10) Pub. No.: US 2013/0217084 A1
(43) Pub. Date: Aug. 22, 2013

(54) PRODUCTION OF OMEGA-3 FATTY ACIDS FROM CRUDE GLYCEROL

(71) Applicant: Zhiyou Wen, Ames, IA (US)

(72) Inventor: Zhiyou Wen, Ames, IA (US)

(21) Appl. No.: 13/788,422

(22) Filed: Mar. 7, 2013

Related U.S. Application Data
(63) Continuation-in-part of application No. 13/486,464, filed on Jun. 1, 2012.
Application No. 13/486,464 is a division of application No. 12/466,653, filed on May 15, 2009, now Pat. No. 8,202,713.

(51) Int. Cl. C12P 7/64

(52) U.S. Cl. CPC C12P 7/6427
USPC 435/134; 435/257.1

(57) ABSTRACT

The present invention relates to various methods to produce a variety of omega-3 fatty acids from various species of alga using crude glycerol as a substrate for algal growth. In one embodiment, the present invention relates to various methods to produced docosahexaenoic acid (DHA) from a Schizochytrium, Phaeodactylum, Thraustochytrid, Ulkenia, and/or Labyrinthulea species of alga. In another embodiment, the present invention relates to various methods to produced eicosapentaenoic acid (EPA) from a Schizochytrium, Phaeodactylum, Thraustochytrid, Ulkenia, and/or Labyrinthulea species of alga. In one instance, the methods of the present invention utilize crude glycerol as at least a portion of the culture medium for the various micro-organisms disclosed herein to enable the production of one or more omega-3 fatty acids. In one embodiment, the crude glycerol of the present invention can be generated from a biodiesel process as a substrate for the production of either docosahexaenoic acid (DHA) or eicosapentaenoic acid (EPA).

### RELATED APPLICATION DATA

The present application is a continuation-in-part and claims priority to U.S. patent application Ser. No. 13/486,464, filed Jun. 1, 2012, entitled “Producing Eicosapentaenoic Acid (EPA) from Bio-Diesel-Derived Crude Glycerol,” which claims priority to and is a divisional of U.S. patent application Ser. No. 12/466,653, filed May 15, 2009, entitled “Producing Eicosapentaenoic Acid (EPA) from Bio-Diesel-Derived Crude Glycerol,” and which issued on Jun. 19, 2012 as U.S. Pat. No. 8,202,713. The entireties of all of the above-related priority documents are hereby incorporated by reference in their entireties herein.

### FIELD OF THE INVENTION

The present invention relates to various methods to produce a variety of omega-3 fatty acids from various species of alga using crude glycerol as a substrate for algal growth. In one embodiment, the present invention relates to various methods to produced docosahexaenoic acid (DHA) from a Schizochytrium, Phaeodactylum, Thraustochytrid, Ulkenia, and/or Labyrinthulea species of alga. In another embodiment, the present invention relates to various methods to produced eicosapentaenoic acid (EPA) from a Schizochytrium, Phaeodactylum, Thraustochytrid, Ulkenia, and/or Labyrinthulea species of alga. In one instance, the methods of the present invention utilize crude glycerol as at least a portion of the culture medium for the various micro-organisms disclosed herein to enable the production of one or more omega-3 fatty acids. In one embodiment, the crude glycerol of the present invention can be generated from a biodiesel process as a substrate for the production of either docosahexaenoic acid (DHA) or eicosapentaenoic acid (EPA).

### BACKGROUND OF THE INVENTION

Docosahexaenoic acid is an omega-3 fatty acid that is a primary structural component of the human brain cerebral cortex, sperm, testicles and retina. It can be synthesized from alpha-linolenic acid or obtained directly from fish oil. However, such methods are expensive, time consuming and/or environmentally questionable. Docosahexaenoic acid (DHA, C22:6, n-3) as well as eicosapentaenoic acid are the principal products of α-linolenic acid metabolism in young men and illustrates the importance of DHA production for the developing fetus and healthy breast milk. DHA is a major fatty acid in sperm and brain phospholipids and in the retina. Dietary DHA may reduce the risk of heart disease by reducing the level of blood triglycerides in humans. Below-normal levels of DHA have been associated with Alzheimer's disease. A low level of DHA is also spotted in patients with retinitis pigmentosa.

Eicosapentaenoic acid (EPA, C20:5, n-3) is an important fatty acid in the omega-3 family based on its medically established therapeutic capabilities against cardiovascular diseases, cancers, schizophrenia, and Alzheimer's disease. However, a microbial-based EPA source has not been commercially available. Fish oil as the main source of EPA has several limitations such as undesirable taste and odor, heavy metal contamination, and potential shortage due to overfishing, variation in seasonal availability of source fish, and cost of production. Thus, it would be highly beneficial to identify and develop new sources to produce EPA.

Biodiesel as an alternative fuel has attracted increasing attention in recent years. In the United States, for example, the annual biodiesel production has increased sharply from less than 100 million gallons prior to 2005 to 700 million gallons in 2008. During the biodiesel production process, crude glycerol is created as a byproduct. In general, for every gallon of biodiesel produced, 0.3 kg of glycerol is produced. With biodiesel production growing exponentially, the market is being flooded with crude glycerol. Some uses for this crude product have been developed (e.g., combustion, composting, anaerobic digestion, or feeding for various animals such as pigs and chickens). Converting crude glycerol into value-added products through thermo-chemical or biological methods is another alternative for utilizing this waste stream. However, the amount of crude glycerol being produced still far exceeds the demand for these uses. Because it is prohibitively expensive to convert and purify the crude glycerol into material that can be used for food, cosmetics, or pharmaceutical industries, biodiesel producers are actively searching for new uses for crude glycerol. There is therefore an ongoing need to discover and develop new methods of using crude glycerol in a constructive manner.

Accordingly, there is a need in the art for an economical and environmentally sensitive method to produce various omega-3 fatty acids such as DHA or EPA.

### SUMMARY OF THE INVENTION

The present invention relates to various methods to produce a variety of omega-3 fatty acids from various species of alga using crude glycerol as a substrate for algal growth. In one embodiment, the present invention relates to various methods to produced docosahexaenoic acid (DHA) from a Schizochytrium, Phaeodactylum, Thraustochytrid, Ulkenia, and/or Labyrinthulea species of alga. In another embodiment, the present invention relates to various methods to produced eicosapentaenoic acid (EPA) from a Schizochytrium, Phaeodactylum, Thraustochytrid, Ulkenia, and/or Labyrinthulea species of alga. In one instance, the methods of the present invention utilize crude glycerol as at least a portion of the culture medium for the various micro-organisms disclosed herein to enable the production of one or more omega-3 fatty acids. In one embodiment, the crude glycerol of the present invention can be generated from a biodiesel process as a substrate for the production of either docosahexaenoic acid (DHA) or eicosapentaenoic acid (EPA).

In one embodiment, the present invention relates to a method of producing a fatty acid-rich biomass from crude waste glycerol, comprising the steps of: (i) providing crude glycerol culture medium that is substantially free of soaps and methanol; and (ii) culturing at least one species of Schizochytrium, Phaeodactylum, Thraustochytrid, Ulkenia, and/or Labyrinthulea in the crude glycerol culture medium under conditions that permit the Schizochytrium, Phaeodactylum, Thraustochytrid, Ulkenia, and/or Labyrinthulea to use glycerol in the crude glycerol culture medium as a carbon source to produce a fatty acid-rich biomass.

In another embodiment, the present invention relates to a method of producing docosahexaenoic acid (DHA), eicosapentaenoic acid (EPA), or a combination thereof from crude waste glycerol, comprising the steps of: (a) providing crude glycerol culture medium; and (b) culturing at least one species of Schizochytrium, Phaeodactylum, Thraustochytrid, Ulkenia, and/or Labyrinthulea in the crude glycerol culture medium under conditions that permit the Schizochytrium, Phaeodactylum, Thraustochytrid, Ulkenia, and/or Labyrinthulea to use glycerol in the crude glycerol culture medium as a carbon source to produce biomass that includes DHA, EPA, or a combination thereof.

In still another embodiment, the present invention relates to a Schizochytrium biomass comprising docosahexaenoic acid (DHA), wherein at least a portion of the biomass and at least a portion of the DHA is produced by Schizochytrium using crude glycerol as a substrate.

In still another embodiment, the present invention relates to a Phaeodactylum biomass comprising eicosapentaenoic acid (EPA), wherein at least a portion of the biomass and at least a portion of the EPA is produced by Phaeodactylum using crude glycerol as a substrate.

### BRIEF DESCRIPTION OF THE DRAWINGS

FIG. 1 is a set of graphs illustrating the cell growth, substrate utilization, DHA and TFA production of the continuous culture Schizochytrium limacinum on crude glycerol with different dilution rates (D) (S₀=90 g/L); with FIG. 1A illustrating the biomass yield and productivity, FIG. 1B illustrating the cell growth yield (Y_{x/s}) and specific substrate utilization rate (qₛ), FIG. 1C illustrating the DHA yield and productivity, and FIG. 1D illustrating the TFA (total fatty acid) yield and productivity. Data are means of three consecutive samples at the steady state (after at least three volume changes), and error bars detail standard deviations;

FIG. 2 is a graph illustrating Correlation of 1/D versus 1/S for estimating μₘ and Kₛ values;

FIG. 3 is a graph illustrating the determination of the maintenance coefficient (m) and true growth yield coefficient (Y_{g}) of Schizochytrium limacinum for growth on crude glycerol in continuous culture;

FIG. 4 is a set of graphs illustrating cell growth, substrate utilization, DHA and TFA production of the continuous culture Schizochytrium limacinum on crude glycerol with different feed crude glycerol concentrations (S₀) (D=0.3 day⁻¹), with FIG. 4A illustrating biomass yield and productivity; FIG. 4B illustrating cell growth yield (Y_{x/s}) and specific substrate utilization rate (qₛ), FIG. 4C illustrating DHA yield and productivity, and FIG. 4D illustrating the TFA (total fatty acid) yield and productivity. Data are means of three consecutive samples at the steady state (after at least three volume changes), and error bars detail standard deviations;

FIG. 5 is an illustration of exemplary batch mode and continuous mode algal growth set-ups;

FIG. 6 is a graph illustrating the pHs of various culture media;

FIG. 7 is a graph illustrating various biomass yields at different crude glycerol concentrations over a time period of multiple days;

FIG. 8 is a graph illustrating the growth rates of an algal species at varying concentrations of crude glycerol;

FIG. 9 is a graph illustrating biomass yield over time for tested carbon dioxide levels;

FIG. 10 is a graph illustrating specific growth rate versus carbon dioxide levels;

FIG. 11 is a graph illustrating pH with respect to carbon dioxide level;

FIG. 12 is a graph illustrating cell growth of the continuous culture Phaeodactylum tricornutum on crude glycerol;

FIG. 13 is a graph illustrating TFA production of the continuous culture Phaeodactylum tricornutum on crude glycerol; and

FIG. 14 is a graph illustrating EPA production of the continuous culture Phaeodactylum tricornutum on crude glycerol.

### DETAILED DESCRIPTION OF THE INVENTION

The present invention relates to various methods to produce a variety of omega-3 fatty acids from various species of alga using crude glycerol as a substrate for algal growth. In one embodiment, the present invention relates to various methods to produced docosahexaenoic acid (DHA) from a Schizochytrium, Phaeodactylum, Thraustochytrid, Ulkenia, and/or Labyrinthulea species of alga. In another embodiment, the present invention relates to various methods to produced eicosapentaenoic acid (EPA) from a Schizochytrium, Phaeodactylum, Thraustochytrid, Ulkenia, and/or Labyrinthulea species of alga. In one instance, the methods of the present invention utilize crude glycerol as at least a portion of the culture medium for the various micro-organisms disclosed herein to enable the production of one or more omega-3 fatty acids. In one embodiment, the crude glycerol of the present invention can be generated from a biodiesel process as a substrate for the production of either docosahexaenoic acid (DHA) or eicosapentaenoic acid (EPA).

The invention provides a cost-effective means to produce useful fatty acids such as the omega-3 polyunsaturated fatty acid docosahexaenoic acid (DHA) or eicosapentaenoic acid (EPA) while, at the same time, addressing the problem of the accumulation of excess crude glycerol. The invention is based on the discovery that alga of the genus Schizochytrium produce DHA from crude glycerol and that alga of the genus Phaeodactylum produce EPA from crude glycerol, in particular from crude glycerol from which soaps and methanol have been removed. According to the invention, at least one strain of Schizochytrium or Phaeodactylum is cultured with waste glycerol under conditions that allow the microorganism to use the waste glycerol as a substrate for the production of various fatty acids of interest, for example DHA or EPA, respectively. The resulting biomass is rich in fatty acids, including DHA or EPA and, after suitable processing (e.g., drying), can be used as a food source or food additive. Alternatively, one or more fatty acids of interest may be isolated from the biomass prior to use.

Exemplary species of Schizochytrium that may be used in the practice of the invention to produce DHA include but are not limited to Schizochytrium limacinum, Schizochytrium mangroveei (see, e.g., Journal of Industrial Microbiology and Biotechnology, 2001; Vol. 27; pp. 199 to 202; Journal of Agricultural and Food Chemistry, 2007; Vol. 55; pp. 2906 to 2910). Exemplary species of Phaeodactylum that may be used in the practice of the invention to produce EPA include but are not limited to Phaeodactylum tricornutum. In another embodiment, any suitable micro-organism from the Schizochytrium family can be utilized in connection with the present invention. A suitable example of such a micro-organism includes, but is not limited to, Schizochytrium sp. In still another embodiment, the present invention utilizes a suitable micro-organism from the Thraustochytrid family, the Ulkenia family, and/or the Labyrinthulea family. In yet another embodiment, a mixture of two or more different species of micro-organisms can be utilized in connection with the present invention for the production of, for example, DHA or EPA.

In another embodiment, the Schizochytrium species used for DHA production is a mutant or transformant strain obtained via classical mutation or molecular biology/genetic engineering. In yet another embodiment, the Schizochytrium limacinum species used for DHA production is a mutant or transformant of Schizochytrium limacinum obtained via classical mutation or molecular biology/genetic engineering. In still another embodiment, the present invention relates to the use of a Pythium species, be it a naturally occurring species or a genetically altered, mutant, and/or transformant species, for the production of EPA. In still another embodiment, the present invention relates to the use of a Pythium Irregulare species, be it a naturally occurring species or a genetically altered, mutant, and/or transformant species, for the production of EPA. In these embodiments, any suitable substrate and/or growth media, or medium, disclosed herein can be utilized in conjunction with the production of EPA from the Pythium species listed above.

In another embodiment, the Phaeodactylum species used for EPA production is a mutant or transformant strain obtained via classical mutation or molecular biology/genetic engineering. In yet another embodiment, the Phaeodactylum tricornutum species used for EPA production is a mutant or transformant of Phaeodactylum tricornutum obtained via classical mutation or molecular biology/genetic engineering.

In still another embodiment, the Schizochytrium, Phaeodactylum, Thraustochytrid, Ulkenia, and/or Labyrinthulea species used for DHA and/or EPA production are mutants or transformant strains obtained via classical mutation or molecular biology/genetic engineering.

The crude waste glycerol that is used to prepare the culture medium in which the Schizochytrium, Phaeodactylum, Thraustochytrid, Ulkenia, and/or Labyrinthulea is cultured may be obtained from any source, one example of which is biodiesel production. Biodiesel is made through a catalyzed transesterification between oils or fats (triglycerides) and an alcohol (usually methanol). Common feedstocks are pure vegetable oil (e.g., soybean, canola, sunflower), rendered animal fats, or waste vegetable oils. The theoretical ratio of methanol to triglyceride is 3:1; which corresponds to having one methanol molecule for each of the three hydrocarbon chains present in the triglyceride molecule, and is equivalent to approximately 12 percent methanol by volume. In practice, this ratio needs to be higher in order to drive the reaction towards a maximum biodiesel yield; 25 percent methanol by volume is recommended. The catalyst can be alkalis, acids, or enzymes (e.g., lipase). The majority of biodiesel produced today is made using an alkali (such as NaOH or KOH) catalyzed reaction because this reaction (1) requires only low temperature and pressure, (2) has a high conversion yield (98 percent) with minimal side reactions and a short reaction time, (3) is a direct conversion to biodiesel with no intermediate compounds, and (4) does not require specific construction materials. The glycerol backbone of the triglyceride remains as a waste product after the reaction is completed.

Crude glycerol generated from biodiesel production is impure and of little economic value. In general, glycerol makes up 65 percent to 8 percent (w/w) of the crude stream. The wide range of the purity values can be attributed to different glycerol purification methods or different feedstocks used by biodiesel producers. For example, Thompson and He (Applied Engineering in Agriculture, 2006; Vol. 22; pp. 261 to 265) have characterized the glycerol produced from various biodiesel feedstocks. The authors found that mustard seed generated a lower level (62 percent) of glycerol, while soy oil had 67.8 percent glycerol, and waste vegetable oil had the highest level (76.6 percent) of glycerol. Any of these preparations may be used to make the crude glycerol culture medium that is utilized in the practice of the invention.

Methanol and free fatty acids (soaps) are the two major impurities contained in crude glycerol. The existence of methanol is due to the fact that biodiesel producers often use excess methanol to drive the chemical transesterification and do not recover all the methanol. The soaps, which are soluble in the glycerol layer, originate from a reaction between the free fatty acids present in the initial feedstock and the catalyst (base) as follows:

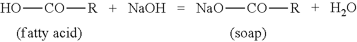

In addition to methanol and soaps, crude glycerol also contains a variety of elements such as calcium, magnesium, phosphorous, or sulfur, as well as potassium and sodium. Cadmium, mercury, and arsenic are generally below detectable limits.

While not wishing to be bound to any one theory, the presence of soaps in the glycerol feedstock tends to inhibit the growth of Schizochytrium, Phaeodactylum, Thraustochytrid, Ulkenia, and/or Labyrinthulea and the production of fatty acids by the microorganism. Therefore, in some embodiments of the invention, soaps are at least partially removed from the crude glycerol prior to culturing the microorganism. Those of skill in the art will recognize that several methods for removing soaps from a liquid are known, and any suitable method may be utilized, so long as the resulting low-soap or substantially soap-free (i.e., less than about 1 percent residual soap) glycerol feedstock is capable of supporting the growth of Schizochytrium, Phaeodactylum, Thraustochytrid, Ulkenia, and/or Labyrinthulea, i.e. so long as other substances or conditions that may be harmful to Schizochytrium, Phaeodactylum, Thraustochytrid, Ulkenia, and/or Labyrinthulea culture are not retained in the low- or no-soap feedstock. For example, the crude glycerol derived from alkali-catalyzed transesterification usually has a dark brown color with a high pH (about 11 to 12). When used in microbial fermentations, crude glycerol is dissolved in a medium solution and the pH is usually adjusted to a neutral range. Under this condition, soaps will be converted into free fatty acids, as shown in the following equation

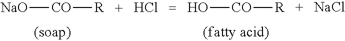

After pH adjustment, the free fatty acids in the crude glycerol stream result in a cloudy solution. After centrifugation, this cloudy solution separates into two clear phases, with the top layer being the free fatty acid phase, and bottom layer the glycerol phase. Thus, soaps may be precipitated from a crude glycerol solution by the addition of, for example, artificial seawater (pH 7.5) (see, e.g., Kester, D. R. et al., Limnology & Oceanography, 1967, Vol. 12; pp. 176 to 179; and Goldman, J. C. and McCarthy, J. J., Limnology & Oceanography, 1978; Vol. 23; pp. 695 to 703); or by the adjustment of the pH to a value that results in conversion of the soaps to fatty acids (e.g., to a pH of at least about 7.5, or further to pH 4 to 4.5 for a complete precipitation of free fatty acids). Once soaps are created, they may be separated and removed from the crude glycerol by any of several suitable means such as by centrifugation and separation of the resulting phases, settling, filtering, straining, etc. In some embodiments, the soaps are collected and reused in other processes.

The presence of methanol in the crude glycerol employed in the invention can also inhibit the growth of Schizochytrium, Phaeodactylum, Thraustochytrid, Ulkenia, and/or Labyrinthulea, and thus methanol may also be removed by any suitable method. For example, due to its relatively high volatility, heating the crude glycerol (e.g., to a temperature of about 100° C. or greater). In some instances, this can be accomplished during sterilization of the crude glycerol, e.g. by autoclaving. In some embodiments, the methanol is evaporated and recaptured for reuse in another process.

Growth conditions for Schizochytrium or Phaeodactylum in the crude glycerol culture medium are studied as described in the Examples below. Generally, the crude waste glycerol is pre-treated prior to culturing by removal of soaps and methanol, as these substances inhibit the growth of Schizochytrium or Phaeodactylum. The removal of soaps and methanol produces substantially soap- and substantially methanol-free crude glycerol, i.e. crude glycerol with less than about 1 percent residual soap or methanol. Herein, the initial glycerol waste (e.g., the waste that is a byproduct of biodiesel production) may be referred to as “crude glycerol” or “crude waste glycerol,” whereas after removal of soaps and methanol, the solution may be referred to as “pre-treated crude glycerol” or “crude glycerol that is substantially free of soap(s) and methanol.” After further preparation (e.g., dilution, supplementation, etc. as described below), a “crude glycerol culture medium” that is used to culture Schizochytrium or Phaeodactylum is produced.

Due to its high glycerol content, crude waste glycerol is a highly viscous liquid and is generally diluted even prior to soap and methanol removal by the addition of an aqueous diluent such as distilled water or artificial sea water. Dilution results in a solution of lower viscosity that is more readily manipulated (i.e., mixed, poured, etc.), and may, depending on the diluent that is used, also lower the pH and cause precipitation of soaps. The decrease in viscosity is important not only for pre-treatment but also later during culturing, when the algal culture must be aerated, sampled, transferred, etc. and high viscosity is detrimental to these processes. The extent of dilution will vary depending on the initial viscosity and glycerol concentration in the crude waste glycerol, which may vary from source to source. Further, dilution may also take into account the optimal amount of glycerol that is to be made available to the Schizochytrium or Phaeodactylum as a substrate in the finally formulated crude glycerol culture medium. Generally, the crude waste glycerol content of the crude glycerol culture medium is from about 10 to about 60 g/L of crude glycerol, or from about 20 to about 50 g/L of crude glycerol, and usually from about 30 to about 40 g/L of crude glycerol. A crude glycerol culture medium with a crude glycerol final concentration (before culturing begins) of from about 30 to about 40 g/L of glycerol generally has the desired properties of (1) being of a suitable viscosity; and (2) being of a sufficiently high concentration to support a Schizochytrium or Phaeodactylum culture to generate desired quantities of fatty acids. 30 g/L of crude glycerol (prior to removal of soaps and methanol) corresponds to an actual “glycerol” content of about 22 g/L (i.e. about 70 percent to 80 percent of crude glycerol is glycerol), since soaps, methanol and other impurities make up about 20 percent to about 30 percent of the initial weight of the crude glycerol. Those of skill in the art will recognize that the actual amount of glycerol in a crude glycerol waste stream may vary from source to source. However, the alga can readily adapt to such relatively minor fluctuations in final glycerol content in the crude glycerol culture medium, for example from about 20 g/L to about 25 g/L.

In addition, other substances essential to the alga growth need to be added to the crude glycerol culture medium. For example, yeast extract may be added to the culture medium in an amount generally ranging from about 1 to about 25 g/L, and usually from about 5 to about 10, 15 or 20 g/L. In most embodiments, the amount of yeast extract in the crude glycerol culture medium will be of a final concentration (i.e., prior to inoculation with Schizochytrium or Phaeodactylum) in the range of from about 5 to about 10 g/L, which is favorable for maximizing the production of the fatty acid DHA or EPA. In another embodiment, other substances essential to the alga growth that may, or can, be added to the crude glycerol culture medium include, but are not limited to, yeast extract, (NH₄)₂SO₄, diammonium phosphate (DAP), urea, NaNO₃, calcium stearoyl-2-lactylate (CSL), or any combinations of two or more thereof. In this embodiment, each of the aforementioned additives can be added to the culture medium individually, or in the aggregate, in an amount generally ranging from about 1 to about 25 g/L, and usually from about 5 to about 10, 15 or 20 g/L. In still another embodiment, any suitable carbon source and/or nitrogen source can be added to the crude glycerol culture medium. Non-limiting examples of such additives include yeast extract, calcium stearoyl-2-lactylate (CSL), monosodium glutamate (MSG), one or more ammonium salts, nitrates, urea, sodium salts, potassium salts, biotin, one or more vitamins, or mixtures of any two or more thereof. In this embodiment, each of the aforementioned additives can be added to the culture medium individually, or in the aggregate, in an amount generally ranging from about 1 to about 25 g/L, and usually from about 5 to about 10, 15 or 20 g/L.

Thus, a basic crude glycerol culture medium for use in culturing Schizochytrium in order to produce fatty acids such as DHA generally includes at least crude glycerol (usually pretreated) at a final concentration of from about 30 to 40 (e.g., about 30) g/L, and about 5 to 10 (e.g., about 10) g/L of yeast extract. The final crude glycerol culture medium generally has a pH in the range of from about 6.0 to about 6.5, is sterile, and has a viscosity that is suitable for culturing and later harvesting Schizochytrium biomass.

Additionally, a basic crude glycerol culture medium for use in culturing Phaeodactylum in order to produce fatty acids such as EPA generally includes at least crude glycerol (usually pretreated) at a final concentration of from about 30 to 40 (e.g., about 30) g/L, and about 5 to 10 (e.g., about 10) g/L of yeast extract. The final crude glycerol culture medium generally has a pH in the range of from about 6.0 to about 6.5, is sterile, and has a viscosity that is suitable for culturing and later harvesting Phaeodactylum biomass.

Various other substances may be advantageously added to the crude glycerol culture medium. Examples of such substances include but are not limited to various salts, buffering agents, trace elements, vitamins, amino acids, etc. However, as described in the Examples section, one advantage of Schizochytrium cultures, or Phaeodactylum cultures, are that these organisms are relatively hardy and do not require additional supplements in order to grow and produce fatty acid-enriched biomass. In some embodiments of the invention however, various oils are added to the crude glycerol culture medium, as these can enhance biomass and thus the overall production level of particular fatty acids such as DHA or EPA. This enhancement is due to oil absorption by the algal cells and elongation of shorter chain fatty acids (e.g., linoleic acid and R-linolenic acid) into longer chain fatty acids (e.g., linoleic acid and alpha-linolenic acid) into longer chain fatty acids (e.g., DHA or EPA). Examples of suitable oils include but are not limited to soybean oil, flaxseed oil, canola oil, linseed oil, and corn oil. Generally, the amount of oil that is added is in the range of from about 0.5 percent to about 4 percent, and usually about 1 percent.

The preparation of fatty-acid enriched Schizochytrium or Phaeodactylum biomass on a commercial scale may be carried out using any suitable industrial equipment, for example tanks or reaction vessels capable of containing volumes of about 10 to 100 m³. Such vessels are generally known to those of skill in the art, and may also comprise, in addition to a means of adding and removing medium, means for, for example, sampling the medium (e.g., to measure pH), means to monitor and adjust the temperature; means to supply gases (e.g., air, oxygen, etc.) to the culture; means to agitate the medium, etc.

### Schizochytrium-Based Fatty Acid Production Examples:

In order to begin an industrial scale culture, a substantially pure Schizochytrium culture is obtained (e.g., from the American Type Culture Collection or another suitable source) and used to initiate growth of Schizochytrium under conditions favorable to growth for several days. For example, in the case of the algal species Schizochytrium limacinum SR21 (ATCC MYA-1381), the cells are maintained in 250-mL Erlenmeyer flasks each containing 50 mL of medium, and incubated at 25° C. in an orbital shaker set to 170 rpm. The medium for the seed culture is artificial seawater containing 10 g/L glucose, 1 g/L yeast extract, and 1 g/L peptone. The artificial seawater contains (per liter) 18 grams NaCl, 2.6 grams MgSO₄.7H₂O, 0.6 grams KCl, 1.0 gram NaNO₃, 0.3 grams CaCl₂.2H₂O, 0.05 grams KH₂PO₄, 1.0 grams Trizma base, 0.027 g/L NH₄Cl, 1.35×10⁻⁴ grams of the vitamin B12, 3 mL of chelated iron solution, and 10 mL of PII metal solution containing boron, cobalt, manganese, zinc, and molybdenum. The pH of the medium is adjusted to about 7.5 to about 8.0 before being autoclaved at 121° C. for 15 minutes. The flask cultures are used as inoculums for the fermenter culture. To start the serial scale-up liquid cultures, a suitable amount of Schizochytrium is first prepared by, for example, by washing the agar surface with distilled water, medium, etc. and the Schizochytrium solution is added to a bench scale container suitable for large scale growth of the organism. The Schizochytrium inoculum contains from about 10⁵ to about 10⁷ alga; cells per liter of culture medium that is inoculated. Then, the culture is “stepped up” gradually by initially inoculating a small volume (e.g., about 1 to about 2 liters) which is subsequently transferred to a larger volume.

During culturing of the Schizochytrium, the medium is agitated and air or oxygen (usually air) is supplied to the growing culture. Agitation may be performed, for example, by shaking or rotating the culture (e.g., at an rpm of about 150 to about 200 rpm, usually about 170 rpm) in a bench scale flask culture or by a means of agitation or stirring such as paddles, propellers, or another suitable mechanism in fermentor culture. In fermentor culture, the fermentor is aerated or oxygenated, usually oxygenated during growth. Generally, the oxygen concentration is maintained at a level of about 10 percent to about 50 percent throughout culturing. Those of skill in the art will recognize that the provision of air or oxygen to the culture may also serve to agitate the culture as the gas is blown into or bubbled through the medium.

Typically, in order to maximize the production of fatty acid-enriched biomass, the culturing of Schizochytrium is carried out in two stages. After inoculation of culture medium with the microorganism, a growth phase is undertaken at a temperature of about 25° C. to about 30° C. in order to encourage the accumulation of biomass. Generally, the culture is maintained at this temperature for a period of from about 4 to about 6 days, and usually for about 5 days. Thereafter, in order to promote the accumulation of fatty acids in the Schizochytrium cells, the temperature is decreased to about 20° C. Culturing continues at this lower temperature for a period of from about 1 to about 3 days, and usually for about 2 days. Thus, the total number of days from initial inoculation to harvesting of the Schizochytrium biomass is typically from about 5 to about 7 days, and usually is about 6 days.

Thereafter, the Schizochytrium biomass is harvested by any of several suitable means and methods that are known to those of skill in the art, for example, by centrifugation and/or filtration. Subsequent processing of the biomass is carried out according to its intended use, for example, by dewatering and drying.

Schizochytrium cultured in a crude glycerol culture medium as described herein produces a biomass that is rich is a variety of fatty acids and may be used in a variety of applications. In some embodiments of the invention, the fatty acid enriched biomass that is produced by Schizochytrium according to the methods of the invention is used “as is” i.e. the fatty acids are not separated or isolated from the biomass prior to use. In such embodiments, the biomass may be collected and used directly (e.g., as a wet algal mass) but will more often first be treated by removing some or most or all of the water associated with the biomass. Thus, the invention also encompasses various forms of fully or partially desiccated (dried) biomass produced by Schizochytrium that is enriched for fatty acids (e.g., DHA) due to having been cultured in the presence of crude glycerol as described herein. Such biomass may be used as a food source or additive to feed a variety of organisms, for example fish (especially fish grown in aquacultural fish “farms”); chickens and other poultry (turkeys, Guinea hens, etc.); cows, sheep, goats, horses, and other domestic animals that are typically raised in a “farm” environment, etc. The biomass may be used as food for or to supplement the diet of any species that in any way benefits from the intake of fatty acids, especially DHA, to their diet. Of special interest may be the feeding of the biomass to laying hens to increase the quality (type) of the fatty acids in eggs, or to increase the amount of desired fatty acids in eggs. Similarly, the biomass may be fed to animals raised as food in order to increase the quality (type) of the fatty acids in meat, or to increase the amount of desired fatty acids in meat. Generally, such desired fatty acids include polyunsaturated fatty acids (PUFAs), and in particular, omega-3 fatty acids such as DHA.

In other embodiments of the invention, the fatty acids, especially DHA, may be separated from the biomass (i.e., substantially purified to varying degrees) and then used as, for example, food supplements. Such fatty acids preparations may contain a mixture of one or more fatty acids originating from the Schizochytrium biomass of the invention, or alternatively, the fatty acids may be isolated to provide one or more substantially pure fatty acids.

The biomass and/or fatty acids prepared according to the methods of the invention may be used for purposes other than for food. For example, various skin preparations, cosmetics, soaps, skin cleansers, lotions, sun screen, hair products and other preparations made be formulated to include either the biomass itself, or one or more fatty acids obtained from the biomass. In particular, various “natural” or “green” products may be prepared and marketed as containing biomass that is “naturally” enriched in valuable fatty acids, and which is ecologically responsible due to its preparation using waste crude glycerol.

Algal Strain, Medium and Subculture Conditions:

The algal species Schizochytrium limacinum SR21 (ATCC MYA-1381) is used. The cells are maintained in 250-mL Erlenmeyer flasks each containing 50 mL of medium, and incubated at 25° C. in an orbital shaker set to 170 rpm. The medium for the seed culture is artificial seawater containing 10 g/L glucose, 1 g/L yeast extract, and 1 g/L peptone. The artificial seawater contained (per liter) 18 grams NaCl, 2.6 grams MgSO₄.7H₂O, 0.6 grams KCl, 1.0 gram NaNO₃, 0.3 grams CaCl₂.2H₂O, 0.05 grams KH₂PO₄, 1.0 grams Trizma base, 0.027 g/L NH₄Cl, 1.35×10⁻⁴ grams of the vitamin B12, 3 mL chelated iron solution, and 10 mL PII metal solution containing boron, cobalt, manganese, zinc, and molybdenum. The pH of the medium is adjusted to about 7.5 to about 8.0 before being autoclaved at 121° C. for 15 minutes. The flask cultures are used as inoculums for the fermenter culture.

Continuous Culture Conditions:

Continuous cultures are performed in a 7.5-L New Brunswick Bioflo 110 fermenter with working volume of 4.5 L at 25° C. Agitation is provided by three turbine impellers. During the cultivation, agitation speed is varied to maintain the dissolved oxygen (DO) level above 50 percent of saturation. Compressed air (about 0.1 vvm) is sparged into the culture through a sterilized air filter. The medium pH is controlled within the range of about 6.5 to about 7.5. A medium containing artificial seawater with 90 g/L crude glycerol and 5 g/L corn steep solid is used in initial batch cultures. After 3 days of batch culture, feed medium is added to the fermenter at various dilution rates (with a feed crude glycerol concentration of 90 g/L) or at various crude glycerol concentrations (with a dilution rate of 0.3 day⁻¹). At the same time, equal volumes of cell suspension are withdrawn from the fermenter. The composition of the feed medium is the same as that for the initial batch cultures except different concentrations of crude glycerol is used. Samples are taken from the fermenter on a daily basis for measuring the cell dry weight. The steady state under each operation condition is considered to have been established after at least three volume changes (the total volume of liquid flowing through the fermenter), with a variation of cell dry weight less than 5 percent. The light/photosynthesis contribution for the algal growth is considered negligible in the continuous culture since an opaque heating blanket is wrapped around the glass vessel, and the cell density was high, and thus, the mutual shading effect is severe.

Preparation of Crude Glycerol Medium:

Crude glycerol is obtained from Virginia Biodiesel Refinery (West Point, Va.). The plant uses a 50:50 (w/w) chicken fat and soybean oil mixture for making biodiesel. The following procedures are used to remove soap from crude glycerol: (i) the glycerol is mixed with distilled water at a ratio of 1:4 (v/v) to reduce the viscosity of the fluid; (ii) the pH of the fluid is adjusted to 3 with sulfuric acid to convert soap into free fatty acids that precipitated from the liquid; (iii) the fatty acid-precipitated liquid is kept static for 30 minutes to allow free fatty acid and glycerol to separate into two phases; (iv), the free fatty acid phase (upper phase) is removed from the crude glycerol phase through a separation funnel; and (v) other medium compositions (seawater salts, corn steep solids, etc.) are added to the glycerol solution to adjust to the desired levels. This glycerol-containing medium is then autoclaved at 121° C. for 15 minutes. It should be noted that it has been shown that autoclaving can drive off methanol from the medium.

Analyses:

A 10-mL cell suspension sample is taken daily from the fermenter and centrifuged at 8000 rpm for 5 minutes. The solids (cell pellets) are rinsed with distilled water, and freeze-dried to obtain the cell dry weight. When the culture reached steady-state, the freeze-dried algal samples are further analyzed for fatty acid composition using the method reported in Pyle et al., Producing Docosahexaenoic Acid (DHA)-Rich Algae from Biodiesel-Derived Crude Glycerol: Effects of Impurities on DHA Production and Algal Biomass Composition, Journal of Agricultural and Food Chemistry, Vol. 56, 2008, pp. 3933 to 3939, while the residual glycerol concentration in the supernatant is measured using a Roche glycerol assay kit (R-Biopharm Inc, Marshall, Mich.).

Results:

Effects of Dilution Rate on Cell Growth and DHA Production:

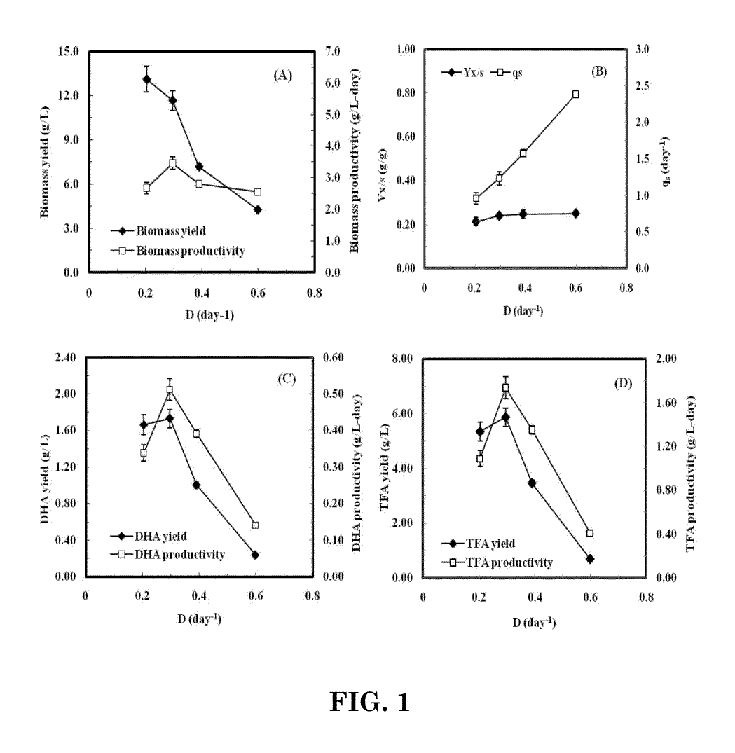

Continuous cultures of Schizochytrium limacinum are first investigated at different dilution rates (D) with a feed crude glycerol concentration of 90 g/L (70.91 g/L true glycerol). As shown in FIG. 1A, the steady-state biomass yield decreases with increasing D from 0.2 to 0.6 day⁻¹, while the highest biomass productivity (ca. 3.46 g/L-day) is obtained at D=0.3 day⁻¹. The cells are washed out when the dilution rate is further increased to around 0.7 day⁻¹. Within the dilution rates investigated, the residual glycerol concentration increases with the increasing dilution rate. Here, one is to assume that the growth of Schizochytrium limacinum follows the Monod equation shown below:

μ=D=(μₘ·S)/(Kₛ+S)

where μ is specific growth rate, μₘ is the maximum specific growth rate, S is the limiting substrate concentration, Kₛ is the half-saturation constant; by inverting the above equation, one can obtain:

(1/D)=(1/μₘ)+(Kₛ/μm)·(1/S).

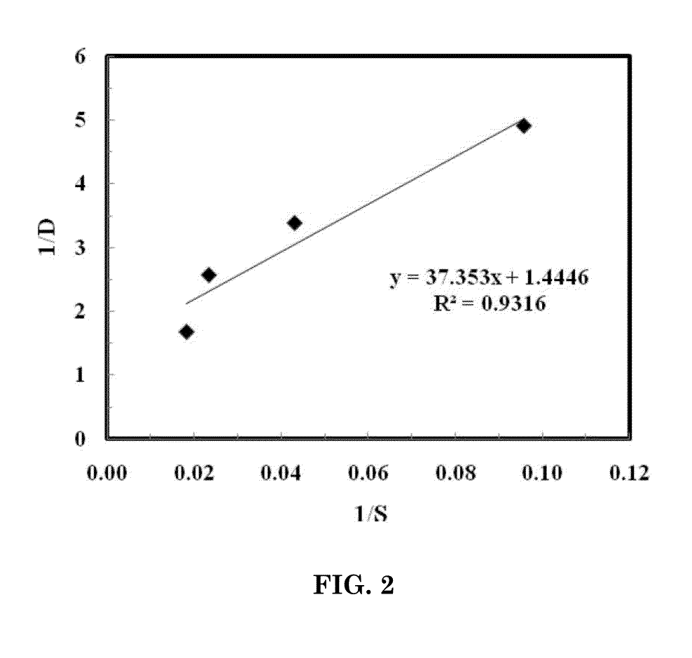

By plotting the 1/D versus 1/S curve (FIG. 2), the value of μₘ and Kₛ are determined to be 0.692 day⁻¹ and 25.87 g/L, respectively.

FIG. 1B illustrates that within the range of dilution rate tested, both the yield coefficient on glycerol (Y_{x/s}) and the specific glycerol consumption rate (qₛ) increased with dilution rate. Such a trend is considered due to the maintenance activities of the algal cells at different dilution rates (i.e., specific growth rate). The dependency of Y_{x/s} on dilution rate can be expressed as:

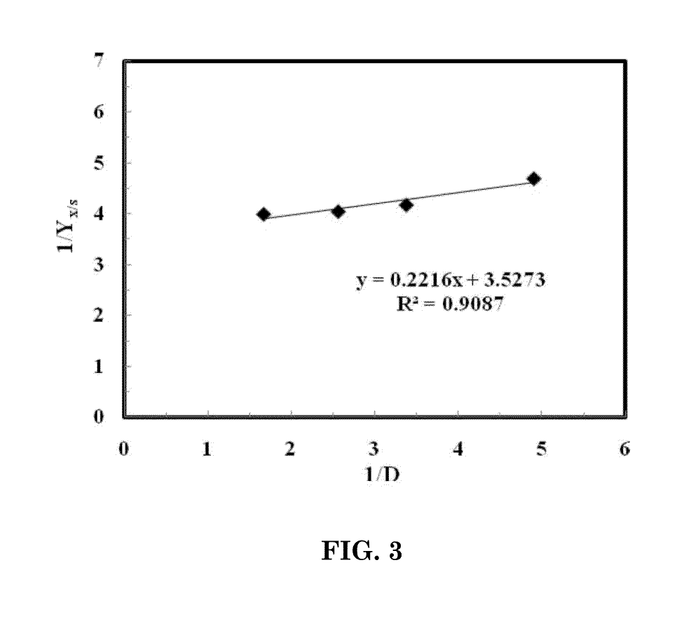

(1/Y_{x/s})=(1/Y_{g})+(m/μ)=(1/Y_{g})+(m/D)

where Y_{g} is the true cell growth yield and m is the maintenance coefficient. By linear regression of 1/Y_{x/s} versus 1/D (FIG. 3), the values of Y_{g} and m are estimated as 0.283 g/g and 0.2216 day⁻¹, respectively.

The fatty acid composition of Schizochytrium limacinum under different dilution rates is presented in Table 1. The algae have a relatively simple fatty acid profile with palmitic acid (C16:0) and DHA being the major fatty acids, and myristic acid (C14:0), stearic acid (C18:0) and docosapentaenoic acid (C22:5) being the minor fatty acids. The percentage of each individual fatty acid (% TFA, total fatty acid) is relatively stable, while the cellular content of TFA and DHA decreases significantly when dilution reaches 0.6 day⁻¹. As far as DHA production is concerned, FIG. 1C shows that the highest DHA yield and DHA productivity are obtained at a dilution rate of 0.3 day⁻¹. The TFA yield and productivity with this dilution rate has a similar trend to those of DHA yield and productivity (FIG. 1D).

TABLE 1

| | | D (day⁻¹) | | | |
|---|---|---|---|---|---|
| Fatty acid | Unit | 0.2 | 0.3 | 0.4 | 0.6 |
| C14:0 | % TFA | 3.28 ± 0.02 | 3.96 ± 0.16 | 4.13 ± 0.09 | 3.25 ± 0.21 |
| C16:0 | % TFA | 57.87 ± 0.44 | 54.61 ± 0.32 | 60.46 ± 1.80 | 53.66 ± 2.62 |
| C18:0 | % TFA | 1.42 ± 0.11 | 3.86 ± 0.17 | 1.37 ± 0.10 | 3.57 ± 0.31 |
| C22:5 | % TFA | 6.38 ± 0.10 | 6.47 ± 0.19 | 5.39 ± 0.36 | 4.47 ± 0.17 |
| C22:6 | % TFA | 31.05 ± 0.47 | 31.09 ± 1.04 | 28.64 ± 1.48 | 35.05 ± 1.34 |
| TFA content | mg/g | 407.17 ± 9.67 | 502.5 ± 6.57 | 481.78 ± 15.90 | 159.87 ± 8.12 |
| DHA content | mg/g | 126.45 ± 4.72 | 148.03 ± 2.85 | 139.34 ± 7.91 | 55.38 ± 2.88 |

Table 1 details the fatty acid composition (% TFA, total fatty acid), and TFA and DHA contents (mg/g DW) of Schizochytrium limacinum at different dilution rates (D) (feed glycerol concentration, S₀, is set at 90 g/L).

Effects of Feed Glycerol Concentration on Cell Growth and DHA Production:

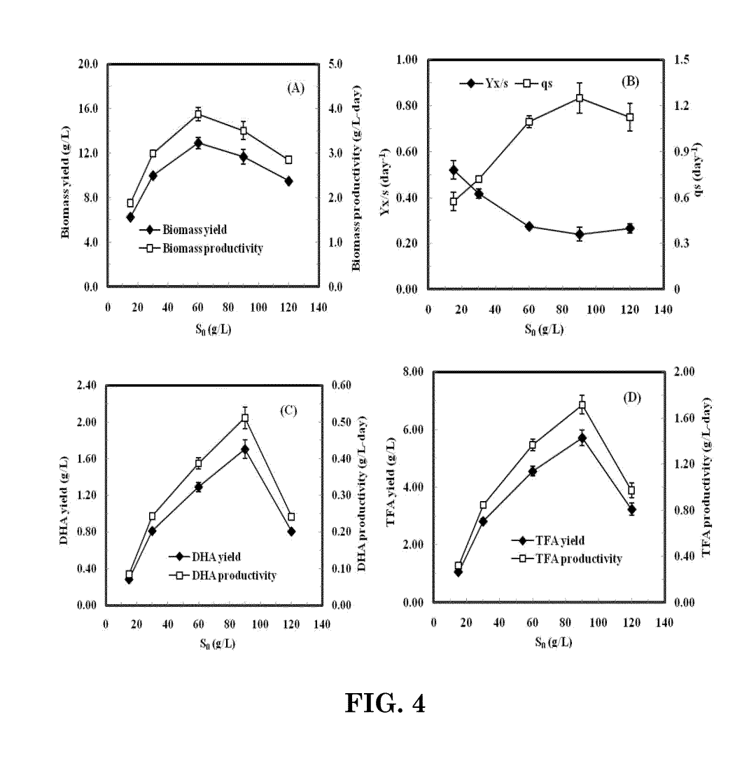

The physiological responses of Schizochytrium limacinum to the change of feed glycerol concentration (S₀) are investigated with a dilution rate of 0.3 day⁻¹. FIG. 4A illustrates that the trend of biomass yield and productivity with S₀ are the same, i.e., both the biomass yield and productivity increased with increasing S₀ from 15 to 60 g/L, and then decreases when S₀ exceeds 60 g/L. At S₀=15 and 30 g/L, the residual glycerol concentration is close to zero, while when S₀ exceeds 30 g/L, certain amounts of residual glycerol exists in the reactor. The changes of Y_{x/s} and qₛ with S₀ are shown in Figure B. Y_{x/s} decreases with the increasing S₀, indicating a more efficient glycerol utilization at lower S₀ levels. Since the qₛ is the quotient of specific growth rate over Y_{x/s}, and the specific growth rate (i.e., dilution rate) are kept constant, the change of qₛ versus S₀ showed a trend opposite that of Y_{x/s} versus S₀ (FIG. 4B).

Table 2 shows that fatty acid composition of Schizochytrium limacinum at different S₀ levels. Overall, the percentage of each fatty acid (% TFA) is maintained stable except that the percentage of C18:0 fluctuated with S₀. The TFA content increased with S₀, with increasing S₀ from 15 to 90 g/L, but decreases when S₀ reaches 120 g/L. The DHA content with S₀ has a similar trend with that of TFA. FIG. 4C shows that the trend of DHA yield with S₀ is the same as that of DHA productivity; the highest DHA yield and productivity are obtained at S₀=90 g/L. With respect to the TFA production, FIG. 3-4D shows that the trend of TFA yield and productivity with S₀ are similar to the DHA yield and productivity with S₀=90 g/L being the optimal level.

TABLE 2

| | | Feed crude glycerol concentration (g/L) | | | | |
|---|---|---|---|---|---|---|
| Fatty acid | Unit | 15 | 30 | 60 | 90 | 120 |
| C14:0 | % TFA | 2.70 ± 0.06 | 3.81 ± 0.05 | 3.88 ± 0.03 | 3.96 ± 0.16 | 3.18 ± 0.02 |
| C16:0 | % TFA | 53.10 ± 0.31 | 57.45 ± 0.35 | 56.29 ± 0.38 | 54.61 ± 0.32 | 57.37 ± 0.15 |
| C18:0 | % TFA | 12.10 ± 0.07 | 3.68 ± 0.27 | 4.95 ± 0.36 | 3.86 ± 0.17 | 9.65 ± 0.63 |
| C22:5 | % TFA | 5.71 ± 0.13 | 6.41 ± 0.06 | 6.51 ± 0.18 | 6.47 ± 0.19 | 4.95 ± 0.13 |
| C22:6 | % TFA | 26.39 ± 0.28 | 28.65 ± 0.21 | 28.37 ± 0.80 | 31.09 ± 1.04 | 24.86 ± 0.33 |
| TFA content | mg/g | 170.27 ± 11.01 | 282.15 ± 8.45 | 352.56 ± 13.26 | 502.5 ± 6.57 | 340.88 ± 12.13 |
| DHA content | mg/g | 44.96 ± 1.98 | 81.20 ± 2.54 | 100.02 ± 4.49 | 148.03 ± 2.85 | 84.79 ± 4.61 |

Table 2 details fatty acid composition (% total fatty acid, TFA) and TFA and DHA contents (mg/g DW) of Schizochytrium limacinum at different feed crude glycerol concentrations (S₀) (D is set at 0.3 day⁻¹).

Comparison of DHA Production with Different Culture Methods:

An overall comparison of cell growth and DHA production obtained by different culture methods is given in Table 3. The biomass yield of the continuous culture is much lower than the batch and fed-batch culture, due to the “dilution” effect as fresh medium is continuously fed to the fermenter. The biomass productivity of the continuous culture is higher than that of batch culture, but lower than the fed-batch culture. The growth yield coefficient on crude glycerol shows that the continuous culture and batch culture had a similar efficiency for utilizing crude glycerol. Table 3 also shows that the DHA content and DHA yield of the algae biomass are lower than both the batch culture and fed-batch culture. In terms of DHA productivity, however, the three-culture modes had a similar level, without significant differences (P>0.05).

TABLE 3

| | | Culture methods | | |
|---|---|---|---|---|
| Parameter | Unit | Batch | Fed-batch^{a,b,c} | Continuous^{d} |
| Maximum specific growth rate | day⁻¹ | 0.685 | Not reported | 0.692 |
| Maximum biomass yield | g/L | 18.04 ± 1.02 | 37.90 | 11.78 ± 0.86 |
| Maximum biomass productivity | g/L-day | 3.06 ± 0.07 | 3.75 | 3.48 ± 0.20 |
| Overall Y_{x/s} | g/g | 0.28 ± 0.02 | Not reported | 0.26 ± 0.01 |
| DHA content | mg/g DW | 170.4 ± 11.2 | 173 | 148.2 ± 2.9 |
| DHA yield | mg/L | 3.07 ± 0.19 | 6.56 | 1.74 ± 0.10 |
| DHA productivity | g/L-day | 0.51 ± 0.04 | 0.56 | 0.52 ± 0.03 |
| Reference | | (Chi et al., 2007) | (Chi et al., 2009) | This work |

^{a}A two-stage DO shifting strategy was used, which DO was shifted from 50% in fermenter culture to flask culture at 40th hour culture time.
^{b}The standard deviations were not reported.
^{c}Pure glycerol was used.
The data listed was corresponding to D = 0.3 day⁻¹, and S₀ = 90 g/L

Comparison of cell growth and DHA production of Schizochytrium limacinum using different culture methods with crude glycerol as a substrate.

Given the data contained above, the DHA production level obtained from crude glycerol culture is comparable to those using glucose or pure glycerol. In addition, the algal biomass derived from crude glycerol contains no heavy metals and has a nutritional quality similar to commercial algae. A crude glycerol-based continuous culture provides quantitative information of the physiological behavior of this species.

In the study of the effects on dilution rate on the cell growth, the cells are washed out when the dilution rate is increased from 0.6 to 0.7 day⁻¹, which is in agreement with maximum specific growth rate (0.692 day⁻¹) determined from the Monod kinetic model. Compared to other continuous alga cultures such as Nitzschia laevis on glucose and Chlamydomonas reinhardtii on acetate, the contribution of maintenance energy (m=0.2216 day⁻¹) to the growth yield (Y_{g}=0.283 g/g) of Schizochytrium limacinum on crude glycerol is rather large, indicating less efficiency of crude glycerol utilization for cell growth.

The phenomenon that high residual glycerol concentration occurs at higher dilution rates and higher S₀ levels are also observed in the continuous culture of other microorganisms. For example, when the diatom Nitzschia laevis is grown at higher dilution rate (D>0.3 day⁻¹) or higher feed glucose region (S₀>20 g/L), the steady-state residual glucose is higher. Similarly, at the respire-fermentative region (i.e., higher dilution rate) of the yeast Saccharomyces cerevisiae, the steady-state sugar concentration is usually high.

The continuous culture is also a better approach to investigate the fatty acid composition of the algae biomass. In a batch culture process, the fatty acids, particularly unsaturated fatty acid, is strongly correlated with the “age” of the cells. Fatty acids accumulated at the stationary phase of a batch culture, but decrease rapidly when the cells transit from stationary phase to the death phase. As a result, precisely identifying an optimal harvest time when the fatty acid content reaches the highest level is difficult. Compared to the batch and fed-batch cultures, the continuous culture provides a stable fatty acid profile at a fixed operational condition (dilution rate and S₀). Indeed, the fatty acid profile (particularly the TFA and DHA content) of the steady-state algal biomass determined herein is very stable, with less fluctuation compared with the batch culture processes.

Continuous culture usually gives a high biomass and end-product productivity in the fermentation process. The results obtained herein show that the biomass productivity is higher than that of batch and fed-batch cultures. In the present invention, the pH-adjusted crude glycerol solution is simply left stationary to separate the soap by gravity; as a result, there is still a certain amount of emulsified soap residues left in the solution. The difference in crude glycerol pre-treatment procedure may yield a reduction in DHA production as the existence of soap is a proven inhibitory for DHA synthesis in the algal culture. Given this, in one embodiment, the crude glycerol of the present invention is further processed to remove as much of the soap compounds contained therein as possible. In one instance, the crude glycerol of the present invention is substantially free of soap, or soap compounds. As used herein, “substantially free of soap” means that the amount of soap remaining in the crude glycerol substrate media is less than about 5 percent by weight, less than about 2.5 percent by weight, less than about 1 percent by weight, less than about 0.5 percent by weight, less than about 0.1 percent by weight, less than about 0.01 percent by weight, less than about 0.001 percent by weight, or even zero percent by weight. Here, as well as elsewhere in the specification and claims, individual numerical values and/or range limits can be combined to form new and/or undisclosed ranges.

In summary, the above results indicate the great potential of producing DHA from biodiesel-derived crude glycerol by algal fermentation.

### Phaeodactylum-Based Fatty Acid Production Examples:

In these EPA-based examples, the alga Phaeodactylum tricornutum (UTEX 640) is used. The algal cells are maintained f/2 medium containing artificial sweater supplemented with (per liter) 0.075 grams NaNO₃, 0.005 grams NaH₂PO₄.H₂O, 0.03 grams Na₂SiO₃.9H₂O, 1 mL of trace metal solution containing iron, copper, sodium, zinc, cobalt, and manganese, and 0.5 mL of vitamin solution containing thiamine HCl, biotin, and cyanocobalamin. Artificial seawater contained (per liter) 18 grams NaCl, 2.6 grams MgSO₄.7H₂O, 0.6 grams KCl, 1.0 grams NaNO₃, 0.3 grams CaCl₂.2H₂O, 0.05 grams KH₂PO₄, 1.0 grams Trizma base, 0.027 g/L NH₄Cl, 1.35×10⁻⁴ grams vitamin B₁₂, 1 mL chelated iron solution, and 10 mL PII metal solution containing boron, cobalt, manganese, zinc, and molybdenum. The medium is autoclaved at 121° C. for 15 minutes. The cells are incubated in 250 mL Erlenmeyer flasks with 50 mL of medium. The flasks are kept on a static shelf with an ambient temperature of 23° C. in static shelf. Illumination is provided by 40-W cool white plus fluorescent lights at 125 μmol s⁻¹ m⁻² measured with an LI-250A light meter and Quantum Q40477 sensor (Li-Cor Biosciences, Lincoln, Nebr., USA). The sub-cultured cells are used as inoculum in the study of mixotrophic culture using biodiesel derived crude glycerol.

Bubble Column Culture System:

Mixotrophic algal culture is performed in glass bubble columns with 50 cm in length and 37 mm in inner diameter. The bottoms of the columns are cone-shaped. The medium for algal culture is f/2 medium supplemented with different concentrations of biodiesel derived glycerol. The medium s autoclaved at 121° C. for 15 minutes. Compressed air (about 1 vvm) is sparged into the bottom of the columns through sterilized air filters. To investigate the effects of CO₂ addition on the algal growth performance, pure CO₂ from a compress tank are mixed with the compress air (at different ratios) through gas flow meters, and mixed gases are then introduced into the bubble columns. The culture system is maintained at 20±1° C. with continuous illumination at 125 μmol s⁻¹ m⁻² through cool white plus fluorescent lights. The working volume of the reactor is controlled at 400 ml.

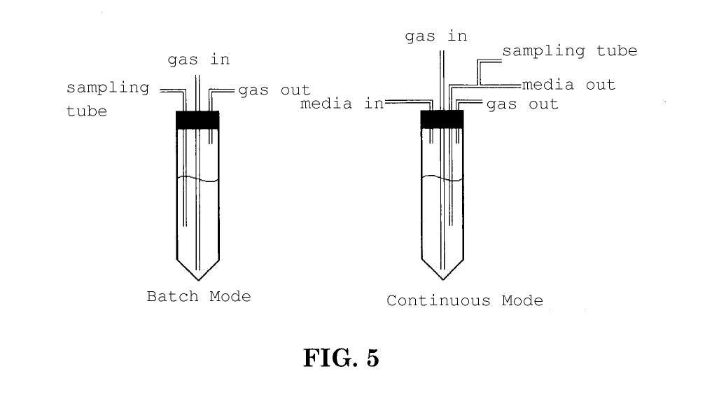

In the continuous culture, a medium containing f/2 medium composition with 0.08 M crude glycerol is used in initial batch cultures. After 6 days of batch culture, feed medium is added to the reactor at various dilution rates. The feed medium composition is identical with those of initial batch culture medium. At the same time, equal volumes of cell suspension are withdrawn from the reactor. Samples are taken from the reactor on a daily basis for measuring the cell density. The steady-state under each operation condition is considered to have been established after at least five consecutive samples with less than 5 percent variation of cell density are achieved. FIG. 5 shows a comparison of the inputs and outputs of both of the modes of operation studied.

Preparation of Crude Glycerol-containing Medium:

The crude glycerol is obtained from Virginia Biodiesel Refinery (West Point, Va.) that produces biodiesel from a 50:50 (w/w) mixture of soybean oil and chicken fat. The procedures of removal soap from crude glycerol and the subsequent preparation of glycerol-containing medium are summarized as follows: (i) the glycerol is mixed with distilled water at a ratio of 1:4 (v/v) to reduce the viscosity of the fluid; (ii) the pH of the fluid is lowered to around 3 with sulfuric acid to convert soap into free fatty acids that precipitated from the liquid; (iii) the precipitated solids formed an upper phase after the liquid is kept static for 30 minutes; and (iv) the free fatty acids in the upper phase are removed from the crude glycerol phase by the means of a separation funnel. This crude glycerol is then added to the f/2 medium at the desired concentration.

Analyses:

Biomass Concentration:

To determine the cell biomass concentration, a relationship between the biomass concentration and optical density (at 440 nm) of the cell culture solution is experimentally established as:

y=1.9029(x)−0.0062

where x is the density of the cells (g/L) and y is the OD. Afterward, a one mL cell culture sample is taken from the bubble columns, measured for its OD value, and converted into biomass concentration using the above equation.

Fatty Acid Analyses:

The cell culture solution harvested at the stationary phase of batch culture or the steady state of continuous culture is centrifuged at 6000 rpm for 5 minutes. The cell pellets are washed twice and then freeze-dried. The preparation of fatty acid methyl esters (FAMEs) from the freeze dried cells and the analyses of fatty acid composition are the same as those described previously (see the Pyle et al., 2008, cited above).

Results:

Batch Mode Culture—Effect of Nitrogen Source:

The effects of the nitrogen source on the growth and fatty acid production of Phaeodactylum tricornutum are studied. The nitrogen source in the f/2 medium is adjusted to contain ammonium chloride, sodium nitrate, and urea. The concentration of each nitrogen source is 11.8 mM of nitrogen. Cell growth performance in nitrogen-free medium is also tested. Cultures are allowed to grow until stationary phase is reached. An overview of cell growth and fatty acid production are shown in Table 4.

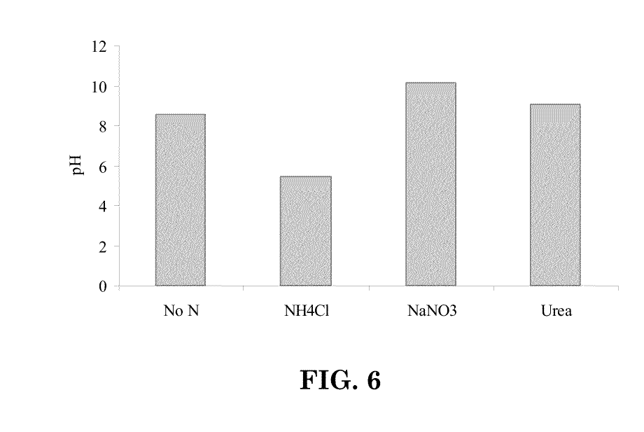

Compared with nitrogen-containing medium, the nitrogen-free medium results in a rather poor biomass yield, specific growth rate and biomass productivity. The poor biomass yield and productivity are expected, because of the role of nitrogen in protein synthesis. Ammonium chloride also has a poor biomass yield and productivity which is likely related to the resulting drop in pH from the ammonium ions (FIG. 6). Sodium nitrate and urea have the best biomass yield and productivity, with sodium nitrate outperforming urea. In terms of fatty acid production, nitrogen-free medium resulted in highest TFA content; however, the TFA yield and productivity are much lower than those from the nitrogen-containing medium. The EPA production from the nitrogen free medium is also lower than those of the nitrogen-containing medium. Among the three nitrogen sources, ammonium chloride results in the highest TFA content, but the lowest TFA yield due to the poor growth from this nitrogen source. The EPA yield and productivity from the sodium nitrate medium is the highest. Collectively, it is found that nitrate is the best of the nitrogen sources for Phaeodactylum tricornutum in term of cell growth and fatty acid production performance, this nitrogen source is used in the following experiments.

TABLE 4

| | | No Nitrogen | NH₄Cl | NaNO₃ | Urea |
|---|---|---|---|---|---|
| Biomass | Maximum | 1.05 ± 0.01 | 1.59 ± 0.27 | 3.50 ± 0.29 | 3.01 ± 0.62 |
| | biomass | | | | |
| | (g/L) | | | | |
| | Specific | 0.122 | 0.213 | 0.339 | 0.292 |
| | growth rate | | | | |
| | (day⁻¹) | | | | |
| | Productivity | 0.087 ± 0.001 | 0.133 ± 0.022 | 0.184 ± 0.015 | 0.158 ± 0.033 |
| | (g/L · day) | | | | |
| TFA | Content | 297.17 ± 29.41 | 158.06 ± 36.44 | 102.64 ± 8.44 | 134.15 ± 31.44 |
| | (mg/g) | | | | |
| | Yield | 311.19 ± 28.05 | 245.63 ± 31.02 | 360.78 ± 56.56 | 416.28 ± 166.14 |
| | (mg/L) | | | | |
| | Productivity | 25.93 ± 2.34 | 20.47 ± 2.59 | 18.99 ± 2.98 | 21.91 ± 8.74 |
| | (mg/L · day) | | | | |
| EPA | Content | 24.00 ± 1.44 | 26.22 ± 4.36 | 24.75 ± 1.39 | 22.19 ± 1.21 |
| | (mg/g) | | | | |
| | Yield | 25.14 ± 1.29 | 42.49 ± 13.88 | 86.49 ± 5.91 | 67.13 ± 16.41 |
| | (mg/L) | | | | |
| | Productivity | 2.10 ± 0.11 | 3.54 ± 1.16 | 4.55 ± 0.31 | 3.53 ± 0.86 |
| | (mg/L · day) | | | | |

Batch Mode Culture—Effect of Crude Glycerol Concentration:

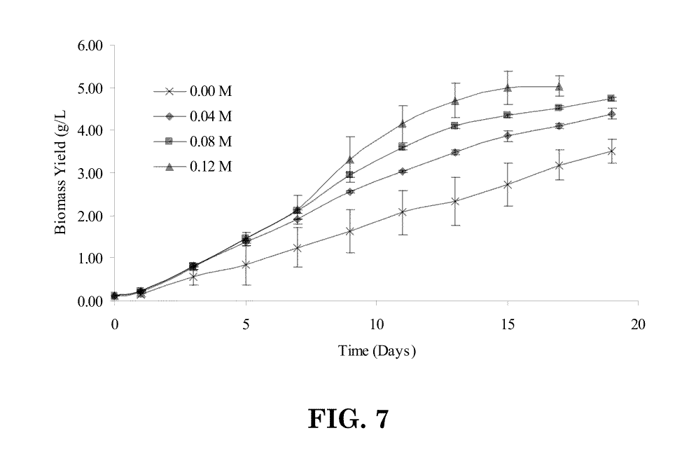

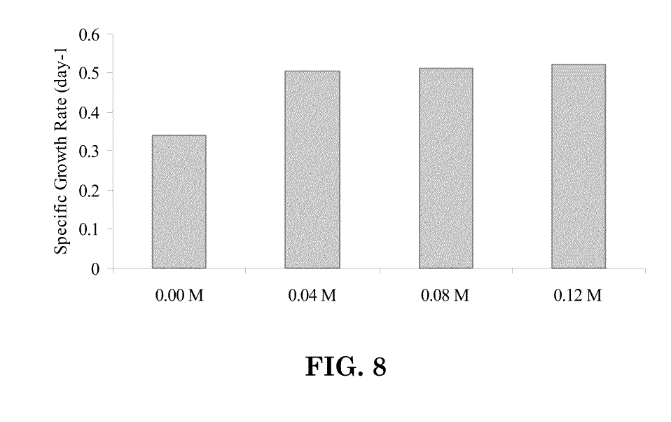

The effects of crude glycerol concentration on the mixotrophic culture of the Phaeodactylum tricornutum is tested at concentrations of 0, 0.04, 0.08, and 0.12 M. FIG. 7 shows that the biomass yield increases with increasing amounts of crude glycerol. As can be seen from FIG. 8, the specific growth rates at 0.04 M, 0.08 M, and 0.12 M of crude glycerol are all very similar. Substrate inhibition can occur after the carbon source is increased to a certain concentration, but this is not observed with the concentrations of crude glycerol tested. Table 5 shows that fatty acid composition and EPA production of the mixotrophic Phaeodactylum tricornutum at different crude glycerol concentrations. The major fatty acid contained in the cells were palmitic acid (C16:0), palmitoleic acid (C16:1), and eicosapentanoic acid (C20:5) with minor amount of myristic acid (C14:0) and oleic (C18:1). For different crude glycerol concentrations, the fatty acid ratios are relatively stable. TFA and EPA content increase with increasing crude glycerol concentration. EPA productivity also increases as crude glycerol concentration increases.

TABLE 5

| | | Concentration | | | |
|---|---|---|---|---|---|
| Fatty acid | Unit | 0.00M | 0.04M | 0.08M | 0.12M |
| C14:0 | % TFA | 7.47 ± 0.58 | 10.08 ± 0.12 | 11.28 ± 0.24 | 13.73 ± 1.50 |
| C16:0 | % TFA | 22.48 ± 0.23 | 21.78 ± 0.42 | 20.60 ± 0.15 | 19.12 ± 0.74 |
| C16:1 | % TFA | 41.63 ± 2.73 | 40.35 ± 0.45 | 39.40 ± 0.42 | 37.99 ± 3.58 |
| C18:1 | % TFA | 4.18 ± 0.43 | 5.51 ± 0.16 | 7.03 ± 0.13 | 8.39 ± 0.22 |
| C20:5 | % TFA | 24.24 ± 2.72 | 22.28 ± 0.59 | 21.69 ± 0.28 | 20.77 ± 2.26 |
| TFA content | mg/g | 102.64 ± 8.44 | 119.32 ± 5.87 | 152.17 ± 4.42 | 167.70 ± 22.12 |
| EPA content | mg/g | 24.75 ± 1.39 | 26.57 ± 1.24 | 33.01 ± 0.93 | 34.50 ± 0.92 |
| EPA yield | mg/L | 86.49 ± 5.91 | 119.57 ± 5.09 | 155.99 ± 3.44 | 173.57 ± 11.87 |
| EPA productivity | mg/L · day | 4.55 ± 0.31 | 6.29 ± 0.27 | 8.21 ± 0.18 | 10.20 ± 0.70 |

Batch Mode Culture—Effect of Carbon Dioxide Level:

Carbon dioxide (CO₂) plays an important role in the mixotrophic culture of Phaeodactylum tricornutum. Providing increased carbon dioxide levels to autotrophically grown algae such as Phaeodactylum tricornutum and Chlorella fusea has been observed to increase lipid content. However, the resulting pH drop from CO₂ dissolving into water to form carbonic acid must be taken into account as algae that require neutral or basic conditions will experience poor growth performance at acidic pH levels.

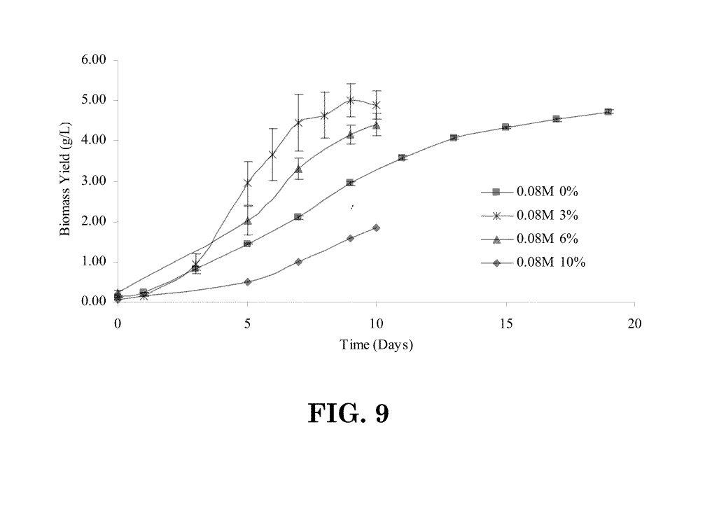

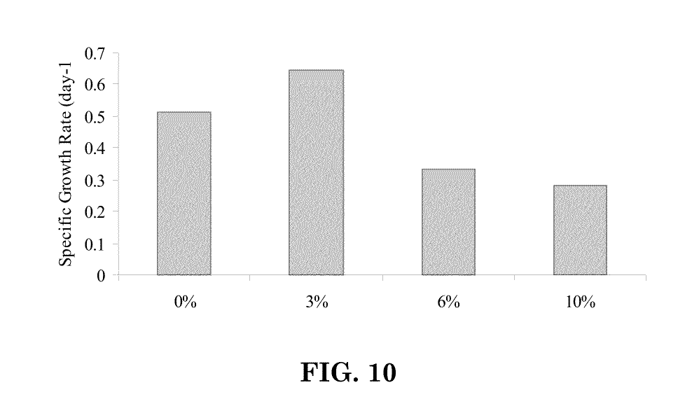

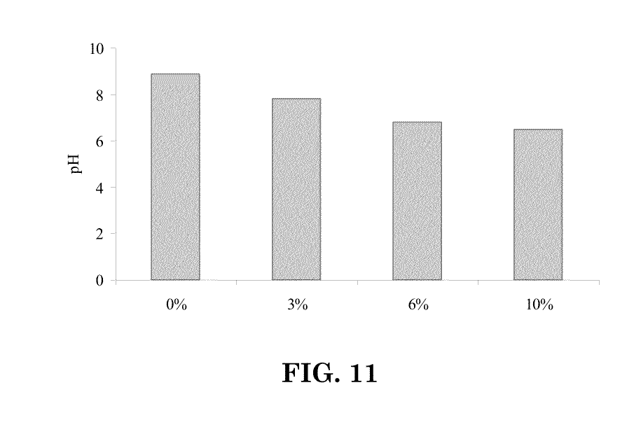

In this study, four levels of CO₂ are tested with Phaeodactylum tricornutum grown in batch mode. As shown in FIG. 9, the highest biomass yield is seen with 3 percent CO₂ supplementation, followed by 0 percent, 6 percent, and 10 percent CO₂ supplementation. Notably, the addition of 3 percent and 6 percent CO₂ results in similar or greater biomass yields in nearly half the time required by 0 percent CO₂. The specific growth rate of Phaeodactylum tricornutum also reaches the highest level at 3 percent CO₂ addition (FIG. 10). The effect of carbon dioxide level on culture pH is shown in FIG. 10. The decreasing specific growth rate at 6 percent and 10 percent CO₂ may be due to the pH drop in the medium (FIGS. 10 and 11).

Table 6 shows a summary of fatty acid analysis data. EPA is present in a consistent ratio among treatments, except for 10 percent CO₂, which causes a small decline. The level of CO₂ addition causes significant fluctuations in the % TFA of myristic acid (C14:0), palmitic acid (C16:0), and palmitoleic acid (C16:1). Myristic acid content increases from 0 percent to 3 percent, but decreases with additional CO₂ supplementation. Palmitic acid content decreases from 0 percent to 3 percent, then increases with further addition of CO₂. Palmitoleic acid content behaves similarly to palmitic acid content, with the same decrease from 0 percent to 3 percent and increasing with additional CO₂. The TFA and EPA contents are fairly similar between treatments. Where the level of CO₂ yields noticeable changes is in the EPA yield and EPA productivity. A level of 3 percent CO₂ results in the best performance in these two measurements. Supplementing with 3 percent and 6 percent CO₂ causes an increase in EPA productivity when compared to 0 percent CO₂ addition. Increasing the supplemented CO₂ to 10 percent caused mostly undesirable effects.

TABLE 6

| | | Level | | | |
|---|---|---|---|---|---|
| Fatty acid | Unit | 0% | 3% | 6% | 10%^{a} |
| C14:0 | % TFA | 11.28 ± 0.24 | 21.41 ± 0.49 | 6.86 ± 0.29 | 6.05 |
| C16:0 | % TFA | 20.60 ± 0.15 | 15.41 ± 0.77 | 25.36 ± 0.44 | 28.53 |
| C16:1 | % TFA | 39.40 ± 0.42 | 33.09 ± 0.19 | 44.43 ± 0.97 | 46.3 |
| C18:1 | % TFA | 7.03 ± 0.13 | 9.37 ± 0.29 | 2.74 ± 0.63 | 1.57 |
| C20:5 | % TFA | 21.69 ± 0.28 | 20.71 ± 0.20 | 20.61 ± 1.20 | 17.54 |
| TFA content | mg/g | 152.17 ± 4.42 | 161.30 ± 5.31 | 159.88 ± 11.28 | 164.47 |
| EPA content | mg/g | 33.01 ± 0.93 | 33.40 ± 0.78 | 32.87 ± 1.13 | 28.85 |
| EPA yield | mg/L | 155.99 ± 3.44 | 169.34 ± 13.08 | 144.92 ± 14.20 | 53.61 |
| EPA productivity | mg/L · day | 8.21 ± 0.18 | 16.93 ± 1.31 | 14.49 ± 1.42 | 5.36 |

^{a}Only one test column of the treatment provided meaningful data.

Continuous Mode Culture:

The batch experiments are used as a basis for parameters selected in continuous culture. The continuous culture is run with 0.08 M crude glycerol, 3 percent CO₂, and sodium nitrate as the nitrogen source. Several dilution rates are investigated under these fixed conditions.

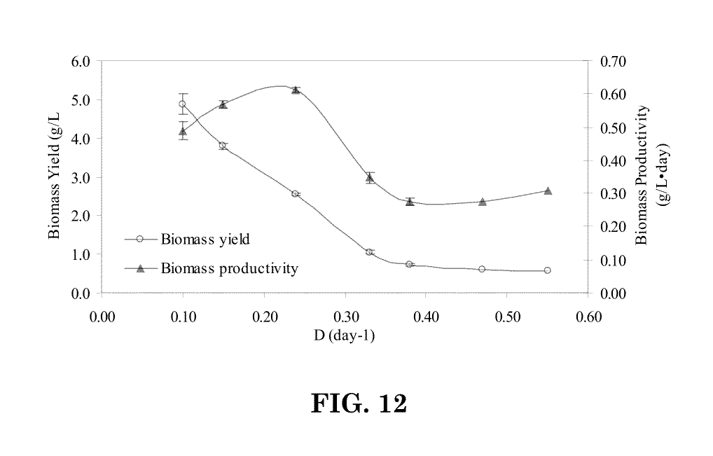

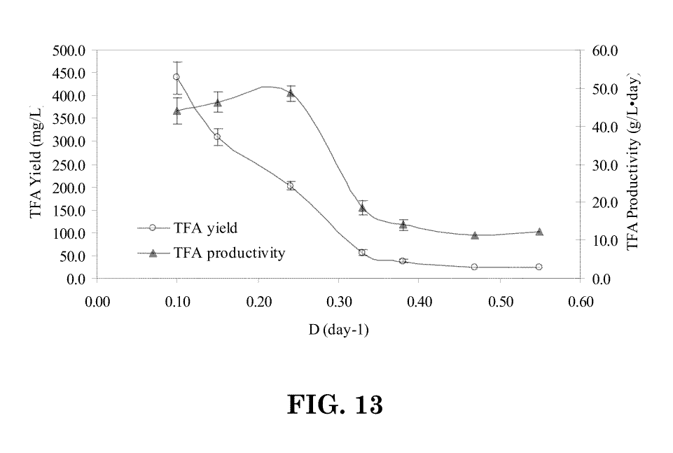

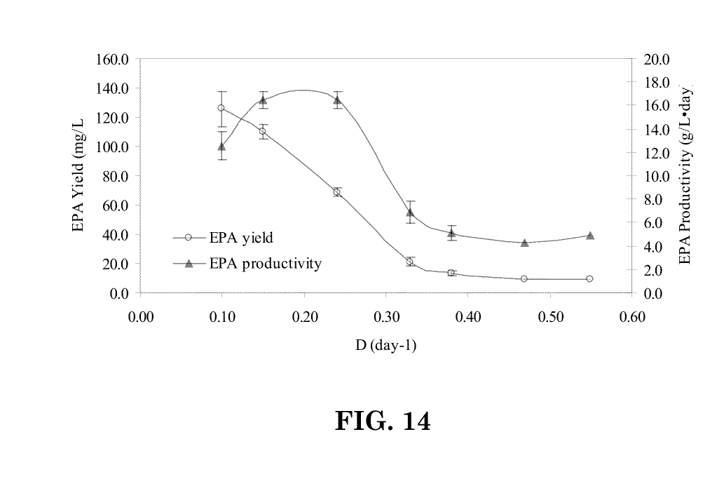

The biomass yields and productivities are shown in FIG. 12. The maximum biomass productivity is observed at a dilution rate of 0.24 day⁻¹. Fatty acid analysis for continuous culture data is summarized in Table 7. There are consistent trends in the increase of palmitic acid (C16:0) content and the decrease of palmitoleic acid (C16:1) content as the dilution rate increases. EPA content is relatively stable for the tested dilution rates, other than the 0.1 day⁻¹ setting, which is slightly lower. FIG. 13 shows the TFA yields and productivities. A dilution rate of 0.24 day⁻¹ leads to the maximum observed TFA productivity. The EPA yields and productivities are shown in FIG. 14. With respect to EPA productivity, dilution rates 0.15 and 0.24 day⁻¹ results in the highest and the values are nearly equal. In FIGS. 12, 13 and 14, the same trend of decreasing yield with a corresponding increase in dilution rate is observed. This trend is expected, as there are less algal cells present with correspondingly higher flow rates of the growth medium.

TABLE 7

| | | Dilution rate (day⁻¹) | | | | |
|---|---|---|---|---|---|---|
| Fatty acid | Unit | 0.1 | 0.15 | 0.24 | 0.33 | 0.38 |
| C14:0 | % TFA | 23.77 ± 0.62 | 23.35 ± 0.75 | 23.72 ± 0.61 | 22.68 ± 0.82 | 19.85 ± 0.47 |
| C16:0 | % TFA | 11.24 ± 0.20 | 11.32 ± 0.54 | 12.75 ± 0.63 | 16.76 ± 1.84 | 20.10 ± 0.46 |
| C16:1 | % TFA | 29.90 ± 0.27 | 25.14 ± 1.20 | 25.85 ± 1.10 | 19.01 ± 0.72 | 19.38 ± 0.51 |
| C18:1 | % TFA | 6.52 ± 0.32 | 4.61 ± 0.41 | 3.76 ± 0.23 | 4.16 ± 0.96 | 4.43 ± 0.10 |
| C20:5 | % TFA | 28.57 ± 0.71 | 35.58 ± 1.63 | 33.92 ± 1.32 | 37.38 ± 3.45 | 36.24 ± 0.59 |
| TFA content | mg/g | 89.84 ± 3.97 | 81.40 ± 4.20 | 79.38 ± 3.37 | 53.30 ± 3.84 | 51.69 ± 6.74 |
| EPA content | mg/g | 25.67 ± 1.41 | 28.93 ± 1.24 | 26.91 ± 1.11 | 19.90 ± 2.00 | 18.76 ± 2.71 |
| EPA yield | mg/L | 125.46 ± 12.41 | 109.67 ± 4.93 | 68.59 ± 3.10 | 20.95 ± 2.85 | 13.48 ± 1.56 |
| EPA | mg/L · day | 12.55 ± 1.24 | 16.45 ± 0.74 | 16.46 ± 0.74 | 6.92 ± 0.94 | 5.12 ± 0.59 |
| productivity | | | | | | |

The batch mode experiments demonstrate the feasibility of using crude glycerol in a mixotrophic culture of Phaeodactylum tricornutum. Of the nitrogen various sources, nitrate leads to the highest EPA productivity. Combining this nitrogen source with supplemental CO₂ and crude glycerol causes further increases in EPA productivity. Continuous mode culture of mixotrophically grown Phaeodactylum tricornutum is more effective than batch mode culture for producing the omega-3 fatty acid EPA. The EPA productivity seen at a dilution rate of 0.24 day⁻¹ is equivalent to or greater than any productivity seen in batch mode, while minimizing the downtime seen with batch mode production. Using crude glycerol in the mixotrophic culture of Phaeodactylum tricornutum creates a value-added product from what is currently an abundant waste product with little value.

While in accordance with the patent statutes the best mode and certain embodiments of the invention have been set forth, the scope of the invention is not limited thereto, but rather by the scope of the attached. As such, other variants within the spirit and scope of this invention are possible and will present themselves to those skilled in the art.

What is claimed is:

1. A method of producing a fatty acid-rich biomass from crude waste glycerol, comprising the steps of:
(i) providing crude glycerol culture medium that is substantially free of soaps and methanol; and
(ii) culturing at least one species of Schizochytrium, Phaeodactylum, Thraustochytrid, Ulkenia, and/or Labyrinthulea in the crude glycerol culture medium under conditions that permit the Schizochytrium, Phaeodactylum, Thraustochytrid, Ulkenia, and/or Labyrinthulea to use glycerol in the crude glycerol culture medium as a carbon source to produce a fatty acid-rich biomass.

2. The method of claim 1, wherein the method utilizes only a species of Schizochytrium.

3. The method of claim 2, wherein the Schizochytrium is Schizochytrium limacinum.

4. The method of claim 1, wherein the method utilizes only a species of Phaeodactylum.

5. The method of claim 4, wherein the Phaeodactylum is Phaeodactylum tricornutum.

6. The method of claim 1, wherein the fatty acid-rich biomass comprises docosahexaenoic acid (DHA), eicosapentaenoic acid (EPA), or a combination thereof.

7. The method of claim 1, wherein Step (i) includes a step of removing soaps and methanol from the crude glycerol.

8. The method of claim 1, wherein the crude glycerol culture medium further comprises one or more oils.

9. The method of claim 8, wherein the one or more oils is selected from flaxseed oil, soybean oil, or combinations thereof.

10. The method of claim 1, wherein crude glycerol is present in the crude glycerol culture medium at a concentration of 30 grams per liter.

11. The method of claim 1, wherein the crude glycerol culture medium further comprises 10 g/L of yeast extract.

12. A method of producing docosahexaenoic acid (DHA), eicosapentaenoic acid (EPA), or a combination thereof from crude waste glycerol, comprising the steps of:
(a) providing crude glycerol culture medium; and
(b) culturing at least one species of Schizochytrium, Phaeodactylum, Thraustochytrid, Ulkenia, and/or Labyrinthulea in the crude glycerol culture medium under conditions that permit the Schizochytrium, Phaeodactylum, Thraustochytrid, Ulkenia, and/or Labyrinthulea to use glycerol in the crude glycerol culture medium as a carbon source to produce biomass that includes DHA, EPA, or a combination thereof.

13. The method of claim 12, wherein the method utilizes only a species of Schizochytrium.

14. The method of claim 13, wherein the Schizochytrium is Schizochytrium limacinum.

15. The method of claim 12, wherein the method utilizes only a species of Phaeodactylum.

16. The method of claim 15, wherein the Phaeodactylum is Phaeodactylum tricornutum.

17. The method of claim 12, wherein the method further comprises the step of pre-treating the crude glycerol to remove soaps and methanol from the crude glycerol.

18. The method of claim 12, wherein the crude glycerol culture medium further comprises one or more oils.

19. The method of claim 18, wherein the one or more oils is selected from flaxseed oil, soybean oil, or a combination thereof.

20. The method of claim 12, wherein crude glycerol is present in the crude glycerol culture medium at a concentration of 30 grams per liter.

21. The method of claim 12, wherein the crude glycerol culture medium further comprises 10 g/L of yeast extract.

22. A Schizochytrium biomass comprising docosahexaenoic acid (DHA), wherein at least a portion of the biomass and at least a portion of the DHA is produced by Schizochytrium using crude glycerol as a substrate.

23. The Schizochytrium biomass of claim 22, wherein the Schizochytrium is Schizochytrium limacinum.

24. The Schizochytrium biomass of claim 22, wherein methanol is removed from the crude glycerol substrate.

25. The Schizochytrium biomass of claim 22, wherein the crude glycerol culture medium further comprises one or more oils.

26. The Schizochytrium biomass of claim 22, wherein the one or more oils are selected from flaxseed oil and/or soybean oil.

27. The Schizochytrium biomass of claim 22, wherein the crude glycerol is present in the crude glycerol substrate medium at a concentration of 30 grams per liter.

28. The Schizochytrium biomass of claim 22, wherein the crude glycerol substrate medium further comprises 10 g/L of yeast extract.

29. The Schizochytrium biomass of claim 22, wherein the crude glycerol substrate is pretreated by removing soaps and methanol from the crude glycerol substrate.

30. A Phaeodactylum biomass comprising eicosapentaenoic acid (EPA), wherein at least a portion of the biomass and at least a portion of the EPA is produced by Phaeodactylum using crude glycerol as a substrate.

31. The Phaeodactylum biomass of claim 30, wherein the Phaeodactylum is Phaeodactylum tricornutum.

32. The Phaeodactylum biomass of claim 30, wherein methanol is removed from the crude glycerol substrate.

33. The Phaeodactylum biomass of claim 30, wherein the crude glycerol culture medium further comprises one or more oils.

34. The Phaeodactylum biomass of claim 30, wherein the one or more oils are selected from flaxseed oil and/or soybean oil.

35. The Phaeodactylum biomass of claim 30, wherein the crude glycerol is present in the crude glycerol substrate medium at a concentration of 30 grams per liter.

36. The Phaeodactylum biomass of claim 30, wherein the crude glycerol substrate medium further comprises 10 g/L of yeast extract.

37. The Phaeodactylum biomass of claim 30, wherein the crude glycerol substrate is pretreated by removing soaps and methanol from the crude glycerol substrate.
